(19) United States
(12) Patent Application Publication
(10) Pub. No.: US 2017/0231753 A1
(43) Pub. Date: Aug. 17, 2017

(54) METHOD AND DEVICE FOR SUSPENSION, LIFTING, AND AUGMENTATION OF THE BREAST, FACE, AND NECK

(71) Applicant: Gilbert W. Lee, San Diego, CA (US)

(72) Inventor: Gilbert W. Lee, San Diego, CA (US)

(21) Appl. No.: 15/430,116

(22) Filed: Feb. 10, 2017

Related U.S. Application Data
(60) Provisional application No. 62/294,181, filed on Feb. 11, 2016.

(51) Int. Cl. A61F 2/12

(52) U.S. Cl. CPC A61F 2/12; A61F 2230/0067; A61F 2210/0004; A61F 2230/0008; A61F 2230/0071

(57) ABSTRACT

The present invention provides a method and device for support of tissues post-surgery, including augmentation and shaping of tissue. The invention provides a non-silicon substrate material which is shaped to support tissue of a subject during the post-surgical recovery period.

### CROSS-REFERENCE TO RELATED APPLICATIONS

This application claims benefit of priority under 35 U.S.C. §119(e) of U.S. Patent Application Ser. No. 62/294,181, filed Feb. 11, 2016, the entire contents of which is incorporated herein by reference in its entirety.

### BACKGROUND OF THE INVENTION

Field of the Invention

The present invention relates generally to surgical methods and more specifically to support of tissues post-surgery.

Background Information

Breast ptosis involves an inferior displacement or sagging of breast parenchyma accompanied by an inferior displacement of the nipple-areolar complex (NAC) with respect to the infra-mammary fold (IMF). Breast ptosis may be found in patients with normal breast size, with relatively small breast size (hypoplasia) or with enlarged breast size (hypertrophy). The combination of ptosis and hypoplasia may be found in the post-partum breast, as the relative breast enlargement of pregnancy and lactation recedes, leaving behind a stretched skin envelope that contains the involuted parenchyma. Hypertrophic breasts typically include some component of breast ptosis in addition to enlarged size.

Breast ptosis is categorized according to the position of the NAC relative to the IMF and relative to the lower pole of breast tissue. Grade 1 ptosis is considered mild, with the nipple just below the IMF but still above the lower pole. Grade 2 ptosis is considered moderate, with the nipple further below the IMF, but with some lower pole tissue below the nipple. Grade 3, severe ptosis, has the nipple well below the IMF, and no lower pole tissue below the nipple. Pseudoptosis, observed with postpartum breast atrophy, includes inferior pole ptosis, with the nipple positioned at or above the IMF.

Mastopexy is a surgical procedure performed to correct breast ptosis. Its goals are to restore the normal contour of the breast mound and to reposition the NAC. To achieve this goal, the excess breast skin is removed or tightened, the parenchymal volume is augmented or reduced as needed, and the NAC is repositioned. The choice of technique is based on the desired final breast size and the degree of ptosis. For minimal ptosis and breast hypoplasia, breast augmentation is sometimes employed in lieu of mastopexy even though it involves the use of a saline or silicone implant. Breast augmentation involves the placement of a breast implant underneath the breast parenchyma or under the pectoralis major muscle, usually through an inframammary fold incision. The implant pushes the parenchyma up and out for a less ptotic and larger appearance.

When more ptosis correction is required, the traditional mastopexy employs NAC repositioning and/or skin resection. A variety of skin incisions may be used, each leaving its own characteristic scar pattern after healing. Skin incisions include the wise or anchor pattern, the circumareolar or periareolar incision, and the vertical pattern which combines a periareolar incision with a vertical breast incision.

Because it is a voluntary, cosmetic procedure, avoiding or minimizing skin incisions adds to the challenge of mastopexy surgery. Mastopexy surgery has traditionally been accompanied by considerable scarring. The extensive scars of the Wise pattern mastopexy, for example, may be necessary if major repositioning and resection is performed, but they represent a significant drawback to the procedure. Though endoscopic techniques have been devised for mastopexy procedures, open surgery is required in many cases to move the nipple, remove excess tissue, or recontour the breast mound. Even if the “anchor” scars of the Wise pattern can be avoided, other open mastopexy techniques still create visible scars on the breast.

The crescent, or periareolar incision may provide for the least noticeable scars; however, serious areola stretching or tissue necrosis is a not infrequent problem as all of the newly lifted parenchymal weight is supported by suture around the areola.

With traditional mastopexy procedures, the tightened skin envelope provides the main support for the lifted breast. Breast implants can be used to fill out the breast contour superiorly, this procedure is termed the augmentation-mastopexy. The augmentation-mastopexy procedure has higher risk of morbidity.

Breast flap repositioning can also be used to fill out the superior contour of the breast; however, this type of breast mound repositioning is a significantly more invasive and complex procedure that involves transposing lower pole tissue bulk and moving it superiorly and securing it to a deeper and higher tissue plane.

Irrespective of the type of mastopexy procedure performed, it is standard procedure that the tightened skin remains the primary support used to keep the breast mound elevated. As the lower pole skin stretches over time as it did in the first instance, ptosis can recur or pseudoptosis (“bottoming out”) can take place. Placement of a breast implant leads to additional weight upon the skin of the lower pole, possibly leading to more rapid skin expansion and recurrent ptosis.

For retaining an upright configuration to the lifted breast and avoiding the post-operative sequelae of recurrent ptosis or pseudoptosis, or “bottoming out”, certain authors have advocated the use of permanent prosthetics such as polypropylene mesh or silicone sheeting to reinforce the lower pole (see, for example, the Orbix breast lifting kit manufactured by Orbix Medical, Tel Aviv, Israel) or wrap the entire parenchyma (see, for example, the Breform™ device, manufactured by Aspide Medical, St. Etienne, France). Concerns about leaving foreign bodies permanently in the breast have limited the widespread adoption of these techniques. A permanent foreign body, according to the literature, is prone to infection and can facilitate a chronic inflammatory reaction. Furthermore, chronic foreign body reactions are often linked to hardening of the tissue and capsular contracture in the same manner as that associated with breast implants. Moreover, a permanent foreign body can interfere with breast cancer surveillance and can distort tissue planes if an oncological procedure is required.

Various permanent sheets such as polypropylene or polyester meshes have been wrapped around the parenchyma (see, for example, the Breform™ device, manufactured by Aspide Medical, St. Etienne, France), but lack an anchoring element that removes load from the skin of the lower pole, thus subjecting the wrapped breast not only to the potential chronic inflammatory response but also to the same ptotic forces as before surgery. Permanent silicone sheets have also been used to cradle the lower pole, which is then suspended from screws in the ribcage, placing all of the load force on one or two fixation points high above the nipple. Additionally, various minimally invasive mastopexy procedures are described in U.S. Pat. No. 7,670,372 to Orbix and Patent Publication No. 2008/0027273 to Gutterman.

More recently, allograft or xenograft products have been proposed as suitable to provide extra support for breast tissue or breast implants during post-mastectomy reconstruction. For example, acellular cadaveric dermal matrix or crosslinked bovine or ovine dermal matrix or collagen have been proposed. Acellular cadaveric dermal matrix has been used extensively in procedures to reinforce the skin flap or reposition the breast during reconstruction procedures. Depending upon the degree of processing involved in the formation of these constructs, they gradually degrade and sometimes resorb, and there is anecdotal evidence that they are replaced with tissue. If degradation occurs too quickly, though, these tissue matrices are not replaced by scar tissue and the patient can suffer recurrent ptosis and “bottoming out.” And if they do not resorb or resorb quite slowly, there is evidence in the literature that they cause increased seroma formation and infection compared to natural tissue flaps. As with the synthetic mesh, these devices require full open procedures and do not alleviate scarring.

A further challenge for mastopexy surgeons is the evolving aesthetic of the upper pole. While traditional mastopexy techniques focused on elevation of the ptotic NAC and lower pole, there is contemporary demand for a fuller look to the upper pole as well. Upper pole fullness, commonly seen following breast augmentation surgery with silicone and shaped implants, has become the paradigm to which some women aspire when thinking about breast surgery. This aesthetic can motivate a mastopexy patient to seek a fuller upper pole in conjunction with a lifted lower pole, a tightened inferior skin envelope and a repositioned NAC.

There remains a need in the art, therefore, for systems and methods of mastopexy that provide the creation or restoration of an uplifted breast shape in a more durable way, as well as preventing post-operative pseudoptosis or recurrent ptosis without compromising the safety or aesthetic quality of the surgical outcome. Desirably, this solution would avoid the potential complications that can accompany the standard mastopexy procedure, permanent mesh placement and/or breast implants while providing long-lasting support for the reshaped breast. For example, it would be desirable to deploy soft-tissue promoting resorbable scaffolds and supports or a long-lasting resorbable matrix that is elastic enough to permit a natural dynamic appearance to the breast via smaller incisions such that, once placed, they offer a good balance between scarring, tissue ingrowth and ultimate resorption. There is a further need in the art for mastopexy techniques that can provide a fuller contour for the upper pole, to satisfy patient aesthetic demands. And, as previously mentioned, avoiding or minimizing scars remains a desirable goal.

### SUMMARY OF THE INVENTION

The present invention provides a method and device for support of tissues post-surgery, including augmentation and shaping of tissue. The invention provides a non-silicon substrate material which is shaped to support tissue of a subject during the post-surgical recovery period.

An one aspect, the invention provides a method of supporting tissue of a breast in a subject. The method includes: a) contacting a flexible, non-silicone material with the breast tissue, the material being configured such that it forms circumferential coverage of the breast tissue for elevation, lifting and support of the breast tissue when attached; b) attaching the material to a position on the pectoral muscle below the breast or to the lowest part of the breast; c) stretching the material upward and attaching the material to a position on the pectoral muscle above the breast; and d) attaching the material to a position on the chest musculature medial and lateral to the breast tissue to offer medial and lateral stability to the breast mound, thereby providing elevation, lifting and support of the breast tissue.

In another aspect, the invention provides a method of supporting, augmenting, and lifting neck tissue in a subject (necklift). The method includes: a) contacting a flexible, non-silicone material with the tissue, the material being configured such that it forms supportive coverage of the tissue for elevation, lifting and support of the tissue when attached; b) attaching the material to a lower position, the lower position being the platysma muscle of the neck; and c) stretching the material upward and attaching the material to an upper position, the upper position being the mastoid periosteum or sternocleidomastoid muscle, thereby providing elevation, lifting and support of the neck tissue.

In another aspect, the invention provides a method of supporting, augmenting, and lifting facial tissue in a subject (facelift). The method includes: a) contacting a flexible, non-silicone material with the tissue, the material being configured such that it forms supportive coverage of the soft tissues of the face for elevation, lifting and support of the tissue when attached, as well as soft tissue augmentation of the face in patients with facial fat atrophy and thin soft tissue coverage; b) attaching the material to a lower position on the face, the lower position being facial subcutaneous fat or a lower portion of the superficial muscular aponeurotic system; and c) stretching the material upward and attaching the material to an upper position, the upper position being deep temporal fascia, orbital rim tissue, or an upper portion of the superficial muscular aponeurotic system, thereby providing elevation, lifting and support of the facial tissue.

In yet another aspect, the invention provides a synthetic or naturally occurring material for supporting and augmenting tissue in a subject in need thereof The material includes a bioabsorbable material being configured such that it provides elevation, lifting, augmentation, and support of tissue when implanted into a subject. In embodiments, the material is shapeable into a 3-D structure for supporting tissue or has a prefabricated shape. Illustrative structures and shapes include generally conical, frustroconical, hemispherical, and teardrop shapes. In some embodiments, the material is TIGR® Matrix, AlloDerm®, Seri® Surgical Scaffold, or Phasix Mesh®.

Various objects, features, aspects and advantages of the inventive subject matter will become more apparent from the following detailed description of preferred embodiments.

### DETAILED DESCRIPTION OF THE INVENTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The present invention provides a method and device for support of tissues post-surgery, including augmentation, lifting and shaping of tissue. The invention provides a non-silicon substrate material which is shaped to support tissue of a subject during the post-surgical recovery period.

The method of the invention may be adapted for supporting or augmenting the shape of tissue in a subject, such as breast, neck and facial tissue.

Generally, the method includes, contacting a flexible, non-silicone material with the tissue of the subject, wherein the material is configured such that it forms supportive coverage of the tissue for elevation, lifting and support of the tissue when attached. The material is then attached to a lower position and then stretched upward and attached at an upper position, thereby providing elevation, lifting, augmentation and support of the tissue.

With regard to supporting breast tissue, the material is attached to a position on the pectoral muscle below the breast (or lower breast tissue) and stretched upward and attach to a position on the pectoral muscle above the breast. It is also attached to the chest musculature medial and lateral to the breast mound providing a circumferential support for the breast mound. As to supporting neck tissue, the material is attached to a lower position, the lower position being the platysma muscle of the neck, and then stretched upward and attached to an upper position, the upper position being the mastoid periosteum or sternocleidomastoid muscle, or supportive structures of the face. As to supporting facial tissue, the material is attached to a lower position, the lower position being facial subcutaneous fat or a lower portion of the superficial muscular aponeurotic system, and then stretched upward and attached to an upper position, the upper position being deep temporal fascia, orbital rim tissue, or an upper portion of the superficial muscular aponeurotic system. In various embodiments, attachment may be via suture, bone screw or any method generally known in the art. The material may also be used in a single piece to support both the face and neck, attaching to the platysma of the neck and SMAS or facial fat below and stretching the material and fixing it above to the mastoid periosteum, and deep supporting structures of the face and neck.

The material may have a prefabricated shape or be custom shaped upon implantation. Generally, for the breast the material may be conical, frustroconical, hemispherical, or teardrop shaped. In one embodiment, the material is in the form of a sheet and shaped upon implantation by providing incisions in the sheet and folding the sheet to produce a 3D-structure configured to provide supportive coverage to tissue. For example, the sheet may include an incision on each of two opposite lateral sides of the sheet and folded and attached in a manner that produces a generally conical shape. For the face and neck, the material may be cut according to template shapes or custom trimmed to accommodate individual facial and neck contours and shapes.

The material may be composed from a variety of materials. In embodiments, the material may include acellular dermal matrix, or a polymer material, such as a mesh. The material may be an absorbable biocompatible material or a permanent material. Ideally materials include TIGR® Matrix, AlloDerm®, Seri® Surgical Scaffold, or Phasix Mesh®, Additionally, the material may be coated with one or more agents that promote collagen formation or ingrowth of cellular matrix in to the material.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the present invention has been described with reference to specific details of certain embodiments thereof, it will be understood that modifications and variations are encompassed within the spirit and scope of the invention. Accordingly, the invention is limited only by the following claims.

What is claimed is:

1. A method of supporting tissue of a breast in a subject comprising:
a) contacting a flexible, non-silicone material with the breast tissue, the material being configured such that it forms circumferential coverage of the breast tissue for elevation, lifting, augmenting and support of the breast tissue when attached;
b) attaching the material to a position on the pectoral muscle below the breast;
c) stretching the material upward and attaching the material to a position on the pectoral muscle above the breast, thereby providing elevation; and
d) attaching the material to the chest musculature medial and lateral to the breast mound providing a circumferential support for the breast mound, lifting and support of the breast tissue.

2. The method of claim 1, wherein the material is a synthetic or naturally occurring, material.

3. The method of claim 2, wherein the material comprises acellular dermal matrix or a polymer material or silk material.

4. The method of claim 2, wherein the material is an absorbable biocompatible material or a permanent material.

5. The method of claim 2, wherein the material is TIGR® Matrix, AlloDerm®, Seri® Surgical Scaffold, or Phasix Mesh®.

6. The method of claim 1, wherein the subjects a human.

7. The method of claim 1, wherein the subject is in need of a breast reconstruction, a breast reduction, a breast lift, or breast augmentation.

8. The method of claim 3, wherein the polymer material is a mesh material.

9. The method of claim 8, wherein the mesh is made from more than one type of polymer.

10. The method of claim 1, wherein the material is attached via sutures.

11. The method of claim 8, wherein the material is in the form of a sheet or the shape of the tissue to be supported.

12. The method of claim 11, wherein the sheet has one or more incisions to allow for sizing of the sheet with the breast.

13. The method of claim 12, wherein the sheet comprises an incision on each of two opposite lateral sides.

14. The method of claim 11, wherein the sheet is from about 5-20 cm by about 10-30 cm.

15. The method of claim 14, wherein the material is from about 10×15 cm or 20×30 cm.

16. The method of claim 8, wherein the material is generally conical, frustroconical, hemispherical, or teardrop shaped.

17. The method of claim 16, wherein the material shape is prefabricated.

18. The method of claim 8, wherein the material comprises fibers that absorb quickly and material that absorbs more slowly over time.

19. The method of claim 1, wherein the material forms complete or incomplete circumferential coverage of the breast tissue.

20. A synthetic or naturally occurring material for supporting tissue in a subject in need thereof, comprising a bioabsorbable material being configured such that it provides elevation, lifting and support of the tissue when implanted into the subject.

21. The material of claim 20, wherein the material comprises acellular dermal matrix, or a polymer material or silk material.

22. The material of claim 20, wherein the material is an absorbable biocompatible material or a permanent material.

23. The material of claim 20, wherein the material is TIGR® Matrix, AlloDerm®, Seri® Surgical Scaffold, or Phasix Mesh®.

24. The material of claim 20, wherein the material is shapeable into a 3-D structure for supporting tissue.

25. The material of claim 24, wherein the material is a sheet with 4 sides, wherein each of 2 opposite lateral sides contain an incision for sizing the material in the subject.

26. The material of claim 24, wherein the material is generally conical, frustroconical, hemispherical, or teardrop shaped.

27. The material of claim 26, wherein the material shape is prefabricated.

28. The material of claim 20, wherein the material is coated with one or more agents that promote collagen formation or cellular ingrowth.

29. A method of supporting, lifting and augmenting neck tissue in a subject comprising:
a) contacting a flexible, non-silicone material with the tissue, the material being configured such that it forms supportive coverage of the tissue for elevation, lifting, augmentation and support of the tissue when attached;
b) attaching the material to a lower position, the lower position being the platysma muscle of the neck; and
c) stretching the material upward and attaching the material to an upper position, the upper position being the mastoid periosteum or sternocleidomastoid muscle or supportive structure of the face, thereby providing elevation, lifting, augmentation and support of the neck tissue.

30. A method of supporting, lifting and augmenting facial tissue in a subject comprising:
a) contacting a flexible, non-silicone material with the tissue, the material being configured such that it forms supportive coverage of the tissue for elevation, lifting and support of the tissue when attached;
b) attaching the material to a lower position, the lower position being facial subcutaneous fat or a lower portion of the superficial muscular aponeurotic system; and
c) stretching the material upward and attaching the material to an upper position, the upper position being deep temporal fascia, orbital rim tissue, or an upper portion of the superficial muscular aponeurotic system, thereby providing elevation, lifting and support of the facial tissue.
